(19) United States
(12) Patent Application Publication
(10) Pub. No.: US 2025/0304459 A1
(43) Pub. Date: Oct. 2, 2025

(54) PROCESS FOR PREPARING AMMONIUM CHLORIDE

(71) Applicant: BASF SE, Ludwigshafen am Rhein (DE)

(72) Inventors: Martin GAERTNER, Ludwigshafen am Rhein (DE); Ralf DIENER, Ludwigshafen am Rhein (DE); Michael SCHREIBER, Ludwigshafen am Rhein (DE); Dieter BAUMANN, Speyer (DE)

(21) Appl. No.: 18/865,392

(22) PCT Filed: May 9, 2023
(86) PCT No.: PCT/EP2023/062165

§ 371 Date: Nov. 13, 2024

(30) Foreign Application Priority Data
May 17, 2022 (EP) 22173680.4

(51) Int. Cl. C01C 1/16; A61K 8/20; A61K 47/02; A61Q 19/00; B01D 9/00

(52) U.S. Cl. CPC C01C 1/164; A61K 8/20; A61K 47/02; A61Q 19/00; B01D 9/0031; B01D 9/0036; B01D 9/0059; B01D 2009/0086; C01P 2004/02; C01P 2006/80

(57) ABSTRACT

A process and a chemical production unit for producing crystalline ammonium chloride in the presence of a crystallization additive are provided, wherein the process comprises the steps a) reacting NH₃ and HCl by feeding NH₃ and HCl to an aqueous solution of ammonium chloride; b) crystallizing ammonium chloride from the aqueous ammonium solution obtained in step a), and c) separating the crystalline ammonium chloride, wherein energy required in step b) is generated in step a). The crystalline ammonium chloride, obtainable by said process, is suitable as a flavoring agent, as an animal feed additive, as an additive for a cosmetic composition or as an additive for a pharmaceutical composition.

The present invention relates to a process for preparing crystalline ammonium chloride from HCl and NH₃ using a crystallization additive, to a chemical production unit for carrying out said process, to a surface-modified crystalline ammonium chloride and to the use thereof as a flavoring agent or as an additive for an animal feed, a cosmetic composition or a pharmaceutical composition.

### BACKGROUND OF THE INVENTION

Ammonium chloride may be produced commercially by several processes, wherein the Solvay process and direct synthesis are the most important ones. The modified Solvay process (ammonium chloride-soda ash process) uses the reaction of ammonia and carbon dioxide in aqueous sodium chloride, wherein ammonium chloride and soda are produced. Alternatively, ammonium chloride can be produced by direct reaction between HCl and NH₃.

DE 2318514 A discloses a process for preparing pulverulent ammonium chloride from gaseous ammonia and HCl gas, wherein the reaction heat is removed by heat exchange. However, the reaction in gas phase usually has some drawbacks, for example, the removal of high energy, undesired deposits in the reactor, and crystal shape and particle size distribution may hardly be controlled.

U.S. Pat. No. 2,024,680 discloses a saturator for producing ammonium chloride comprising a closed chamber, a vessel arranged within and opening at its upper end into said chamber, wherein HCl and NH₃ are fed as gaseous reactants into the vessel at different levels into a solution of NH₄Cl at 65-95° C. The reaction heat liberated during reaction is absorbed as heat of vaporization of NH₃ supplied to the reaction or of any vaporizable water supplied as diluent.

The process for preparing ammonium chloride may also use an aqueous solution of ammonium chloride, derived from various sources or production lines.

CN 103785263 A discloses a process for treating ammonia-containing tail gas generated by calcining ammonium para-tungstate, wherein said tail gas is condensed at 30-80° C. and absorbed by HCl, to form a saturated ammonium chloride solution. A crystalline product may be obtained after evaporative crystallization for further use, for example, in fertilizer production.

CN 103303942 A discloses a process for recovering ammonium chloride from a glycine-containing mother liquor, wherein the mother liquor is subjected twice to a heating/evaporation stage and a flash evaporation stage obtaining a liquid of a concentration of about 45%, followed by continuous crystallization, thickening and centrifugal separation. The process is described as having a low steam consumption.

CN 109437250 A discloses a co-production process of potassium nitrate and ammonium chloride, wherein the ammonium chloride process contains a crystallization section including a flash evaporator and two crystallizers.

CN 107746066 A discloses a process for absorbing acidic gas generated in an ash plasma melting processing system, wherein HCl gas enters an ammonia water spay tower to form a NH₄Cl solution, which is concentrated and crystallized by using steam generated in a waste heat recovery unit of the plasma melting processing system.

A. W. Bamforth et al., Chem. Process Engng. 53(2), 1972, 72-74, discloses a process carried out in aqueous solution, wherein ammonia gas is fed into the conical section of a saturator while HCl diluted with air is passed into the NH₄Cl suspension. The reaction occurs at about 80° C., under reduced pressure, and with excess of NH₃ (pH 8) to produce a supersaturated solution. When there are sufficient crystals in suspension extraction is commenced at a rate corresponding to the amount of ammonium chloride made by the reaction. The suspension is drawn off from the base of the saturator and thickened in hydrocyclones, ammonium chloride is separated in a centrifuge and dried. The mother liquor is recycled to the saturator, and the waste gases from the separator are scrubbed with water.

The known processes often provide technical-grade qualities of crystalline ammonium chloride with no dedicated or low requirements regarding crystal form or particle size, which are usually sufficient for use as a nitrogen fertilizer, as solid electrolytes in dry cell batteries or as a component of fluxes in tin and zinc plating. However, the use as a flavoring agent in food applications or as an additive for an animal feed, a cosmetic composition or pharmaceutical composition requires a higher quality, which may only be achieved with high effort.

The aqueous ammonium chloride solutions are usually subjected to cooling crystallization or preferably evaporation crystallization. The latter uses generally several continuous evaporation units to utilize the applied energy as good as possible, so-called multiple effect evaporation units. The first effect is heated by steam, the following stages are heated by the vapors of the upstream unit. With the number of effects, the steam consumption may be reduced accordingly.

For example, CN 109381880 A discloses a process for treating ammonium chloride-containing wastewater using a three-effect evaporative crystallization system and a condensation device. The evaporative crystallization system contains a crystallization device for vapor-liquid separation, a circulating pump and a heating device, wherein the vapor is used for heating another heating device and condensed.

The reaction and crystallization stages of the preparation of ammonium chloride from an aqueous solution, known in the art, are usually not linked with respect to their energy balance. A high energy input is still necessary when approximately two third of water have to be evaporated from an about 35 wt % solution of ammonium chloride at a temperature of 50° C. This is generally done by external sources, for example, by applying heating steam.

Furthermore, the crystal form should be considered, when using crystalline ammonium chloride in various applications. The crystal form obtained from an aqueous solution may be affected by other substances. Without so-called crystallization additives ammonium chloride crystals grow anisotropically, and dendritic crystals are obtained from pure aqueous solutions. However, dendritic crystals are undesirable with respect to unfavorable flow characteristics, increased residual moisture due to a high specific surface area as well as with respect to caking due to crystals which may become intermeshed.

A. Chianese et al., Journal of Crystal Growth, 166, 1996, 1099-1104, describe the influence of inorganic cations, like Mn(II) ions on the crystal habit. Also, organic substances, like urea or pectin may influence the crystal growth. Ammonium chloride crystals grow in form of cubes in the solution with added urea, as described by Y. V. Naidich, Adgeziya Rasplavov i Paika Materialov (1993), 30, 43-6. Dependent on the amount of pectin in the aqueous solution the octahedral faceting of dendrites may be altered to cubic, as described by I. YA. Melik-Gaikazyan et al., Izvest. Tomsk. Politekh. Inst., 1958, 95, 372-377.

Hence, there is still a need for a process for preparing and crystallizing ammonium chloride which fulfills the requirements of desired product properties and energy consumption, especially on industrial scale.

It is therefore an object of the invention to provide a process for preparing crystalline ammonium chloride in an economical process considering environmental aspects as well. The process should require essentially no or low input of energy, allow for a high yield of crystalline material, especially a minimum loss of starting materials and product loss via wastewater or exhaust air, allow for a targeted regulation of the product properties and/or be reliable with respect to disruptions, for example, due to undesired deposits.

Further, an object of the invention is to provide a crystalline ammonium chloride of high quality suitable to be used as a flavoring agent, an animal feed additive, or an additive for a cosmetic or pharmaceutical composition. The product should have at least one of the following properties, like a defined crystal morphology, a narrow particle size distribution, low residual moisture, good flowability and/or low tendency to caking, even on long storage.

### SUMMARY OF THE INVENTION

It has now been found that crystalline ammonium chloride may be obtained by a two-stage production process, wherein the reaction stage and crystallization stage are substantially separated, but energetically linked. This allows an essentially autothermic process, wherein energy generated in the reaction stage enables evaporation during the crystallization stage.

Accordingly, in a first aspect, the invention relates to a process for producing crystalline ammonium chloride in the presence of a crystallization additive, the process comprising the steps
a) reacting NH₃ and HCl by feeding NH₃ and HCl to an aqueous NH₄Cl solution;
b) crystallizing ammonium chloride from an aqueous NH₄Cl solution obtained in step a), and
c) separating the crystalline ammonium chloride,
wherein the energy required in step b) is generated in step a).

In a further aspect, the invention relates to a surface-modified, crystalline ammonium chloride, obtainable or obtained by a process, as defined in any aspect herein.

In a further aspect, the invention relates to the use of a crystalline ammonium chloride, obtainable or obtained by a process, as defined in any aspect herein, as a flavoring agent, as an animal feed additive, as an additive for a cosmetic composition or as an additive for a pharmaceutical composition.

In a further aspect, the invention relates to a process of applying crystalline ammonium chloride, obtainable or obtained by a process, as defined in any aspect herein, as a flavoring agent to a food or as an additive to an animal feed, to a cosmetic composition or to a pharmaceutical composition.

In a further aspect, the invention relates to a chemical production unit for carrying out a process, as defined in any aspect herein, the chemical production unit comprising
a reaction zone comprising
   an inlet means for feeding NH₃ into the reaction zone,
   an inlet means for feeding HCl into the reaction zone,
   a mixing device,
   a device for phase separation for obtaining an aqueous NH₄Cl solution and a reaction vapor,
   a circulating pump,
   a heat exchanger,
   a means for passing the aqueous NH₄Cl solution to the crystallization zone,
   a means for passing the reaction vapor to the crystallization zone,
a crystallization zone comprising
   an inlet means for feeding the aqueous NH₄Cl solution into the crystallization zone,
   an evaporator for obtaining an aqueous NH₄Cl-containing suspension,
   a circulating pump,
   a heat exchanger,
   a means for passing the NH₄Cl-containing suspension to the separation zone,
a separation zone comprising
   one or more separating devices for obtaining crystalline NH₄Cl, and
an inlet means for feeding a crystallization additive.

### DETAILED DESCRIPTION OF THE INVENTION

The term “essentially autothermic manner”, as used herein, means that during a steady operation the energy required in step b) is generated in step a) to at least 80%, preferably to at least 90%, more preferably to at least 95%.

The term “steady operation”, as used herein, means the standard operating conditions of manufacture of crystalline ammonium chloride thus excluding any transitory periods of start-up or stop of the chemical production unit itself. The addition of water to steps a) to c) should be at a minimum, for example, at most 5% and requires a HCl concentration of at least 37 wt %.

The term “reaction loop”, as used herein, means the loop comprising the devices used for step a), usually comprising a device for phase separation, a circulation pump, a heat exchanger and a mixing device.

The term “crystallization loop”, as used herein, means the loop comprising the devices used for step b), usually comprising an evaporator, a circulation pump and a heat exchanger.

The term «reaction stage», as used herein, means the process within the reaction loop of step a) and the transfer of the reaction vapor and the aqueous NH₄Cl solution, withdrawn from the reaction loop, to the crystallization loop.

The term “crystallization stage”, as used herein, means the process within the crystallization loop of step b) and the transfer of the aqueous NH₄Cl suspension, withdrawn from the crystallization loop, to the separating device.

The term “separation stage”, as used herein, means the process within the separation step c).

The term “reaction zone”, as used herein, means the zone for carrying out the reaction stage.

The “crystallization zone”, as used herein, means the zone for carrying out the crystallization stage.

The “separation zone”, as used herein, means the zone for carrying out the separation stage.

The d₅₀ value in the cumulative frequency distribution of the weight-averaged size distribution function (median diameter, particle size distribution), as is obtained by sieve analysis, indicates that 50 wt % of the particles have a diameter, which is the same as or smaller than the respectively indicated value. The d₅₀ value may be determined according to the method described in ISO 2591-1:1988-12.

The pressure in bar, as used herein, means the pressure in bar absolute.

The term “a combination thereof” or “any combination thereof”, as used herein, means any possible combination of two or more components mentioned in the respective list, either of the same or different kind of components.

As used herein, the indefinite article “a” comprises the singular but also the plural, i.e., an indefinite article in respect to a component of a composition means that the component is a single compound or a plurality of compounds. If not stated otherwise, the indefinite article “a” and the expression “at least one” are used synonymously.

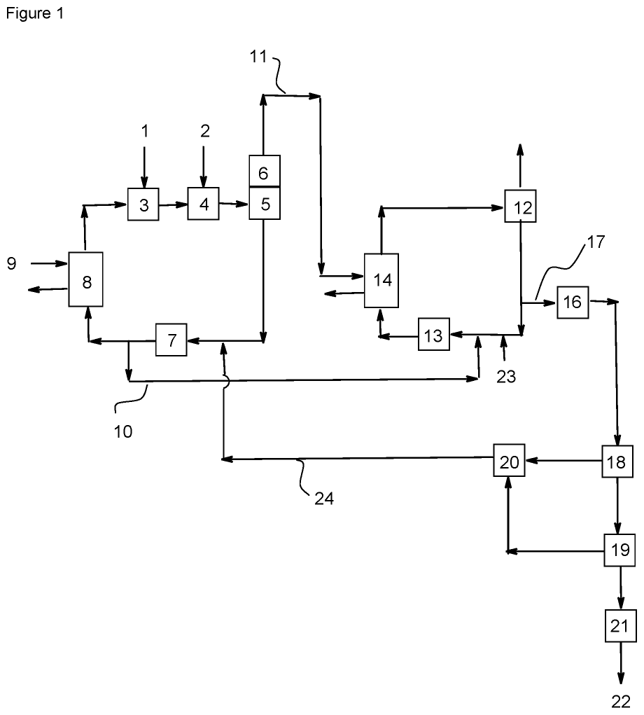

FIG. 1 shows a schematic overview of a preferred embodiment of the process of the present invention.

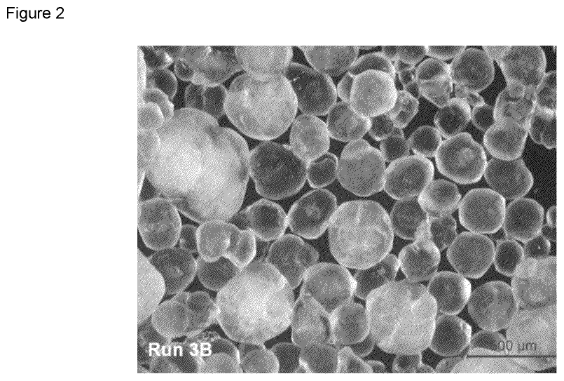

FIG. 2 shows a microscope picture of the product obtainable by the instant process.

The instant process for producing crystalline ammonium chloride comprises mainly three stages: a reaction stage, a crystallization stage and a separation stage.

Process step a) relates to the reaction stage, i.e., a step of reacting NH₃ and HCl, wherein NH₃ and HCl are fed to an aqueous NH₄Cl solution. The aqueous NH₄Cl solution is preferably run through a reaction loop. NH₃ is preferably fed as gas. HCl is preferably fed as an aqueous solution.

Typically, the reaction loop contains a device for phase separation, a pump, preferably a circulating pump, a heat exchanger and a mixing device. The aqueous ammonium chloride solution is circulated within the reaction loop.

Accordingly, in a preferred aspect, the invention relates to a process for producing crystalline ammonium chloride in the presence of a crystallization additive, the process comprising the steps
a) reacting NH₃ and HCl by feeding NH₃ and HCl to an aqueous NH₄Cl solution;
b) crystallizing ammonium chloride from an aqueous NH₄Cl solution obtained in step a), and
c) separating the crystalline ammonium chloride,
wherein the energy required in step b) is generated in step a), and the aqueous NH₄Cl solution is run through a reaction loop, preferably comprising a circulating pump, a mixing device and a vessel for phase separation.

The device for phase separation may be any vessel suitable for phase separation, for example, a phase separator. The heat exchanger may be a tube bundle heat exchanger. The mixing device may be any mixer suitable for pipes, for example, a static mixer, a jet mixer or an in-line mixer.

The heat exchanger may be supplied with an external source of vapor or fuel gas, for example, external vapor with a pressure of from 0.2 to 5 bar, preferably from 0.5 to 3 bar, to heat the system of the reaction zone to a predetermined operating temperature of the aqueous NH₄Cl solution, when the chemical production unit, preferably the reaction zone, running the process, is started. The operating temperature of the aqueous NH₄Cl solution is such at which a steady-state operation of the unit for producing NH₄Cl may be effected. Any condensate from the heat exchanger may be collected in a separate vessel.

Usually, a stream of an aqueous solution of ammonium chloride is run, preferably pumped, through the reaction loop. The concentration of the aqueous solution of ammonium chloride is generally slightly undersaturated, for example, in the range of from 30 to 45 wt %, usually dependent on the operating temperature and pressure in the reaction loop.

The concentration is preferably adjusted to a concentration of about 3 to 10 wt % less than the saturation limit. Preferably, the concentration is about 35 to about 45 wt %, preferably at an operating temperature of about 50 to 130° C., preferably 80 to 120° C., and a pressure of 0.1 to 3 bar within the reaction loop. These ranges of NH₄Cl concentration, pressure and temperature usually includes the pressure and temperature within the whole reaction loop. An especially preferred concentration of the aqueous NH₄Cl solution in the reaction loop is about 35 to 42 wt % at an operating temperature of from 95 to 120° C. and a pressure of 0.2 to 2 bar, in particular 0.2 to 1.5 bar.

The circulating volume of the aqueous solution of ammonium chloride in the reaction loop and the dosing rate of gaseous ammonia are usually such that ammonia is dissolved completely.

The circulating volume of the aqueous solution of ammonium chloride may be varied in a broad range, generally dependent on the production capacity.

The dosing of ammonia gas may be carried out by a suitable inlet means suitable for gases. Suitable examples include any nozzle inlet, a jet pump or a feed pipe, for example, an ejector. The inlet means for dosing ammonia gas is generally located between the heat exchanger and the inlet means for HCl. The dosing rate of ammonia gas may be widely varied, usually dependent on the circulating volume and the production capacity.

The aqueous solution of hydrochloric acid to be fed has preferably a concentration of 20 to 38 wt %, more preferably of 25 to 38 wt %, especially 30 to 38 wt %, and in particular 32 to 38 wt %. The dosing rate of the aqueous solution of HCl may be widely varied, usually dependent on the circulating volume and the production capacity.

Accordingly, in a preferred aspect, the invention relates to a process for producing crystalline ammonium chloride, wherein NH₃ is fed as a gas and HCl is fed as an aqueous solution having a concentration of from 20 to 38 wt %, preferably having a concentration of from 25 to 38 wt %, more preferably having a concentration of from 30 to 38 wt %.

In a further preferred aspect, the invention relates to a process for producing crystalline ammonium chloride, wherein the aqueous NH₄Cl solution in step a) has a concentration of from 30 to 45 wt %. This range of concentration usually includes the concentration of NH₄Cl within the whole reaction loop.

Further preferred is a process for producing crystalline ammonium chloride, wherein step a) is conducted under a pressure of 0.1 to 3 bar and a temperature of from 50 to 130° C. These ranges of pressure and temperature usually includes the pressure and temperature within the whole reaction loop.

Especially preferred is a process for producing crystalline ammonium chloride, wherein the aqueous NH₄Cl solution in step a) has a concentration of from 35 to 42 wt %, in particular at a temperature of from 90 to 120° C. and a pressure of 0.2 to 2 bar.

The aqueous solution of hydrochloric acid may be fed into the reaction loop via a suitable inlet means, for example, using a dosing pump. The aqueous solution of HCl may be fed prior to or into a mixing device, preferably into a mixing device, wherein the neutralization reaction of NH₃ and HCl takes place. The neutralization reaction is usually fast and complete when the aqueous NH₄Cl solution leaves the mixing device.

The amounts of NH₃ and HCl are usually equimolar. The mole ratio of HCl:NH₃ is typically in the rage of from 0.95:1.05 to 1.05:0.95, preferably 0.96:1.04 to 1.04:0.96, more preferably 0.98:1.02 to 1.02:0.98.

Accordingly, in a preferred aspect, the invention relates to a process for producing crystalline ammonium chloride, wherein the mole ratio of HCl:NH₃ is of from 0.95:1.05 to 1.05:0.95, preferably 0.96:1.04 to 1.04:0.96.

Preferably, the pH value of the aqueous NH₄Cl solution of step a) is usually <6, preferably <5 at the operating temperature and pressure.

During the reaction a slight overpressure of, for example, >1 to 3 bar, preferably >1 to 2 bar, may be maintained in order to perform the reaction of ammonia and hydrochloric acid without boiling of the aqueous NH₄Cl solution and formation of vapor within the mixing unit. The complete exothermic energy of the neutralization reaction only leads to an increase in temperature of the circulated aqueous NH₄Cl solution. The operating temperature of the circulated aqueous NH₄Cl solution (reaction solution) may raise in the range of from 5 to 30° C., but preferably not higher than 130° C.

Accordingly, in a preferred aspect, the invention relates to a process for producing crystalline ammonium chloride, wherein the energy required in step b) is generated in step a) by the heat of reaction of reacting NH₃ and HCl, which is released in form of reaction vapor.

Preferably, the reaction step a) comprises a step of reacting NH₃ and HCl and a step of generating reaction vapor, which are carried out subsequently.

Accordingly, in a preferred aspect, the invention relates to a process for producing crystalline ammonium chloride in the presence of a crystallization additive, wherein step a) contains
a-1) a step of reacting NH₃ and HCl, and
a-2) a step of generating reaction vapor,
wherein said steps are carried out separately.

The reaction solution, i.e., the overheated, aqueous NH₄Cl solution generated after neutralization of step a-1), is then passed into the device for phase separation of step a-2), wherein the pressure is usually reduced when entering, for example, to a pressure <1 bar, preferably ≤0.8 bar, more preferably of from 0.2 to 0.8 bar. This may be effected by a conventional means, for example, via a throttle valve or an orifice. The pressure reduction usually leads to boiling of the reaction mixture, wherein water is partially evaporated to generate an aqueous NH₄Cl solution, usually having an increased concentration, and reaction vapor. The obtained aqueous NH₄Cl solution is thereby cooled by some degrees, for example, by 3 to 10° C., for example, to a temperature of about 70 to 120° C.

Preferably, the step a-1) of reacting NH₃ and HCl is carried out in the mixing device and the step a-2) of generating the reaction vapor is carried out in a vessel of phase separation.

The step a-2) usually is a phase separation, wherein reaction vapor and a phase of an aqueous NH₄Cl solution are generated, said solution has usually a higher concentration than the aqueous solution, as obtained after the step a-1).

In a preferred aspect, the invention relates to a process for producing crystalline ammonium chloride, wherein the reaction vapor is generated by a pressure reduction, preferably to a pressure <1 bar, more preferably to a pressure of from 0.2 to 0.8 bar.

Accordingly, further preferred is a process for producing crystalline ammonium chloride in the presence of a crystallization additive, wherein step a) contains
a-1) a step of reacting NH₃ and HCl, at a pressure of >1 to 3 bar, preferably >1 to 2 bar, and
a-2) a step of generating reaction vapor, at a pressure of <1 bar, preferably 0.2 to 0.8 bar,
wherein said steps are carried out separately.

The amount of water to be evaporated is usually calculated such that the concentration of the aqueous NH₄Cl solution within the reaction loop is in the range corresponding to the concentration present within the reaction loop, thus, generally of from 30 to 45 wt %, dependent on the operating temperature and pressure in the reaction loop.

NH₄Cl does not crystallize during step a) within the reaction loop. This enables that the material of the reaction zone may be protected from any abrasion.

The amount of water evaporated in the device for phase separation is usually dependent on the production capacity.

The reaction vapor generated may be purified, especially released from any entrained solution, like NH₄Cl solution, ammonia and/or HCl. Any suitable separating droplet devices may be applied at the top of the device for phase separation, preferably a demister, like an aerosol separator or a droplet separator. Preferably, a combination of an aerosol separator and a droplet separator is used.

Accordingly, in a preferred aspect, the invention relates to a process for producing crystalline ammonium chloride, wherein the reaction vapor is purified.

The reaction vapor may then be passed to the shell side of a heat exchanger of the subsequent crystallization stage of step b).

The aqueous NH₄Cl solution is circulated in the reaction loop, usually via the suction side of a circulation pump and a heat exchanger to the unit, where ammonia gas is generally fed into the reaction loop.

A part of the aqueous NH₄Cl solution is generally removed at a constant rate from the reaction loop and fed to the crystallization loop for further evaporation and crystallization. This happens preferably on the pressure side of the circulation pump. The amount of the circulating volume removed for evaporation and crystallization may be about 2 to 30% of the initial circulating volume, more preferably 5 to 20%, usually dependent on the production capacity.

Accordingly, in a preferred aspect, the invention relates to a process for producing crystalline ammonium chloride, wherein a part of the aqueous NH₄Cl solution obtained in step a) is fed to step b).

Process step b) is the crystallization stage, preferably comprising an evaporative crystallization. The crystallization stage may comprise one or several crystallization stages, preferably one to three crystallization stages, either in parallel or series, preferably in parallel.

Accordingly, in a preferred aspect, the invention relates to a process for producing crystalline ammonium chloride, wherein the crystallization in step b) is conducted by one or more evaporative crystallization loops, preferably arranged in parallel.

The crystallization and evaporation of the ammonium chloride solution is usually carried out in a circulating evaporator. The crystallization loop generally contains a circulating pump, a heat exchanger and an evaporator. The principle of an evaporative crystallization is usually known in the art.

Any suitable evaporator may be used, for example, a Robert-type evaporator, an Oslo-type evaporator or a Forced Circulation (FC) evaporator. The heat exchanger may be a tube bundle heat exchanger.

Crystallization and evaporation may be carried out with a circulation volume which is preferably higher than the circulation volume of the reaction loop. The temperature and pressure are lower than the temperature and the pressure of the reaction loop. This allows that the process of step a) and step b) may be operated in an essentially autothermic manner.

The aqueous NH₄Cl solution, partly removed from the reaction stage, is usually fed into the crystallization loop on the suction side of the circulation pump, preferably via one or preferably more inlet means. The circulating stream may be passed to the heat exchanger, wherein the aqueous NH₄Cl solution is heated. The heat exchanger is operated with the reaction vapor or a part thereof in case of more crystallization loops, withdrawn from the reaction stage. Any condensate is usually collected in a separate vessel.

The process steps a) and b) are generally operated in an essentially autothermic manner during steady operation. The energy to heat the partly removed aqueous NH₄Cl solution from step a) is provided by the reaction vapor generated in step a). Generally, during steady operation the energy required in step b) is generated in step a) to at least 90%, preferably to at least 95%, more preferably to at least 98%. Especially, the reaction vapor is sufficient to process step b) without external energy. In case external energy needed, external vapor, for example, with a pressure of from 0.1 to 3 bar, preferably from 0.2 to 2 bar, may be added to the reaction vapor.

Water is typically evaporated from the solution in the evaporator in an amount sufficient to lead to supersaturation of the solution and crystallization of ammonium chloride and growth of ammonium chloride crystals, which is usually dependent on the production capacity. The pressure and the temperature in the evaporator are usually lower than in the device for phase separation. The resulting aqueous NH₄Cl suspension preferably has a concentration of about 5 to 40 wt %.

The required vacuum for evaporation may be generated by usual means, for example, with a vacuum pump, like a liquid ring compressor or a jet stream pump.

The vapor stream resulting from evaporation may be purified before condensing, especially released from any entrained solution, for example, by any suitable separating device. The optionally purified crystallization vapor may be condensed using a heat exchanger, for example, a plate heat exchanger, and collected in a vessel for condensate. Alternatively, the crystallization vapor may be used for further processes, like pre-heating of raw materials or downstream evaporating devices.

A part of the aqueous NH₄Cl suspension obtained in the evaporator may be constantly removed, usually with a pump, from the crystallization loop and fed to the third stage for separating the ammonium chloride as solid. This may happen between the evaporator and the circulating pump or between the circulating pump and the heat exchanger of the crystallization loop, preferably between the evaporator and the circulating pump. Preferably, the aqueous NH₄Cl suspension, removed from crystallization loop, has a concentration of about 5 to 40 wt %.

Process step c) is the separation stage, preferably a solid-liquid separation stage. The separation step may be carried out with usual separating devices, preferably in more steps, applying energy known in the art for inorganic crystalline salts.

Typically, the separation step is carried out by centrifugation or filtration, like continuous filtration using, for example, a rotary drum filter, preferably by centrifugation. A suitable separating device may be a pusher centrifuge. Optionally, a hydrocyclone may be further used prior to a centrifuge.

Accordingly, in a preferred aspect, the invention relates to a process for producing crystalline ammonium chloride, wherein the crystalline ammonium chloride is separated in one or more separation stages, preferably conducted by centrifugation.

The streams of the one or more evaporators may be fed to the separation stage as separated streams or may be suitably fed as combined streams.

The streams of the evaporator(s) may be passed into a first solid-liquid separation stage using one or more hydrocyclones to separate into a thickened lower flow and an upper flow having a lower solids content. The thickened lower flow may then be passed to a second separation stage using one or more centrifuges.

In case of more hydrocyclones they are preferably used in parallel, wherein the streams are divided into the number of applied hydrocyclones. The thickened lower flows may be combined prior to the second separation stage or treated individually. Preferably, the thickened lower flows are combined for the second separation stage.

The upper flow is usually fed into a container collecting the mother liquid, preferably after combining the upper flows in case of more hydrocyclones.

After centrifugation, wet ammonium chloride may be separated as centrifugate and may be dried in a suitable dryer, for example in a flash dryer, a belt dryer or a drying drum, and cooled.

The saturated ammonium chloride solution separated as centrate may be discharged into a container collecting the mother liquor.

The mother liquor collected from the separation stage may be recycled, preferably pumped, into the reaction loop to close the complete 3 stages. The recycling step enables that the aqueous NH₄Cl solution in the reaction loop may be kept undersaturated.

Accordingly, in a preferred aspect, the invention relates to a process for producing crystalline ammonium chloride, wherein a mother liquid obtained in step c) is recycled to step a).

In order to obtain a desired morphology of the crystalline NH₄Cl, the instant process is carried out in the presence of a crystallization additive. The crystallization additive may be added in different steps of the process, for example, in the crystallization stage and/or separation stage, preferably in step b) and/or step c), preferably step b).

Accordingly, in a preferred aspect, the invention relates to a process for producing crystalline ammonium chloride, wherein the crystallization additive is added in step b) and/or step c), preferably step b).

The crystallization additive may be added to the crystallization stream, before entering the crystallization loop. The crystallization additive may also be added to the crystallization loop of step b), preferably on the suction side of the circulation pump. The crystallization additive may also be added to the separation stage of step c), preferably before entering the centrifuge or preferably before the second separation stage.

The crystallization additive may be added by a suitable inlet means, for example, with a dosing pump or any nozzle inlet.

The crystallization additive may be added to the instant process in any suitable form, preferably as an aqueous solution, for example, in a solution of water, a solution of ammonium chloride like a lean mother liquid obtained in the process, or a solution in ammonia. The solvent of the aqueous solution may be suitably selected dependent on the kind of crystallization additive. Preferably, the crystallization additive is added in form of an aqueous solution, more preferably as a solution in water or in the NH₄Cl-containing mother liquid.

The aqueous solution of the crystallization additive has preferably a concentration of from 0.01 to 10 wt %, based on the total weight of the solution, more preferably 0.1 to 5 wt %. The concentration may be dependent on the kind of crystallization additive and its solubility in the respective aqueous system.

The crystallization additive preferably comprises at least one hydrophilic polymer, more preferably a hydrophilic polymer based on sugar derivatives, most preferably a polysaccharide or a derivative thereof.

The crystallization additive may also be used as any mixture of two or more polysaccharides or derivatives thereof.

Accordingly, in a preferred aspect, the invention relates to a process for producing crystalline ammonium chloride, wherein the crystallization additive is a polysaccharide, a derivative thereof or any combination thereof.

The polysaccharide or a derivative thereof is advantageously selected such that not only the polysaccharide or a derivative thereof itself but also any thermal breakdown products, in the amounts typically present or formed, are suitable as additives for food or animal feed as well as and do not adversely affect the taste of any food processed with the instantly produced crystalline ammonium chloride. It is preferred to use a polysaccharide of natural origin or one formed by modification of a natural polysaccharide with a neutral taste and with approval under food law.

The polysaccharide or a derivative thereof may be a heteropolysaccharide, a derivative thereof, a homopolysaccharide or a derivative thereof.

Heteropolysaccharides may be selected from alginic acid, salts or derivatives thereof, like sodium alginate, potassium alginate, ammonium alginate, calcium alginate, propane-1,2-diol alginate, agar, carrageenan, processed Eucheuma algae, locust bean gum/carob gum, guar gum, gum tragacanth, gum arabic, xanthan gum, karaya gum, tara gum, gellan gum, konjac gum, konjac glucomannan, soybean-hemicellulose, pectin or a derivative thereof like amidated pectin, and any combination thereof.

The heteropolysaccharides are typically obtained by fermentation or by isolation from natural sources.

The homopolysaccharides are usually homopolysaccharides based on glucose and derivatives thereof, preferably cellulose or a derivative thereof.

Accordingly, in a preferred aspect, the invention relates to a process for producing crystalline ammonium chloride, wherein the crystallization additive is selected from a heteropoly-saccharide, a derivative thereof, cellulose, a derivative thereof and any combination thereof.

Suitable cellulose derivatives are, for example, cellulose ethers, on which hydroxyl groups are partially or completely substituted by ether groups. The cellulose ether may be an alkyl ether or an arylalkyl ether, which alkyl or arylalkyl groups may be further substituted by hydroxy, carboxy or carboxylate groups. Corresponding counterions for carboxylate groups may be an alkali metal ion, such as sodium or potassium, or an ammonium ion. Cellulose ethers may carry one type of substituent on the cellulose ether molecular chain, while mixed ether may carry two or more different substituents, like methylhydroxyethyl cellulose.

Preferred cellulose derivatives are methyl cellulose, ethyl cellulose, propyl cellulose, carboxymethyl cellulose (CMC), hydroxyethyl cellulose, hydroxypropyl cellulose, methylhydroxyethyl cellulose, methylhydroxypropyl cellulose, methylhydroxybutyl cellulose, ethylhydroxyethyl cellulose, carboxymethyl hydroxyethyl cellulose, benzyl cellulose and any mixed cellulose ethers.

Among the carboxymethyl celluloses, the sodium compound is preferred.

The cellulose ethers are prepared in a known way, typically by the action of alkyl halides or arylalkyl halides, epoxides or activated olefins on cellulose that has been activated with bases, for example, with an aqueous sodium hydroxide solution. Cellulose ethers are usually commercially available, for example, under the trade name Tylose® or Tylopur® (for food applications).

Preference is given to a polysaccharide or a derivative thereof, which already has approval as a food additive.

Examples of a heteropolysaccharide include alginic acid (E 400), sodium alginate (E 401), potassium alginate (E 402), ammonium alginate (E 403), calcium alginate (E 404), propane-1,2-diol alginate (E 405), agar (E 406), carrageenan (E 407), processed Eucheuma algae (E 407 a), locust bean gum/carob gum (E 410), guar gum (E 412), gum tragacanth (E 413), gum arabic (E 414), xanthan gum (E 415), karaya gum (E 416), tara gum (E 417), gellan gum (E 418), gum ghatti (E 419), konjac gum (E 425i), konjac glucomannan (E 425ii), soybean-hemicellulose (E 426), pectin (E 440i), amidated pectin (E 440ii) and any combination thereof.

Examples of a homopolysaccharide or a derivative thereof include cellulose (E 460), methyl cellulose (E 461), ethyl cellulose (E 462), hydroxypropyl cellulose (E 463), hydroxypropyl methyl cellulose (E 464), ethyl methyl cellulose (E 465), carboxymethyl cellulose (E 466), cross-linked sodium carboxymethyl cellulose (E 468), enzymatically hydrolyzed carboxymethyl cellulose (E 469), polydextrose (E 1200), pullulan (E 1204), oxidized starch (E 1404), monostarch phosphate (E 1410), distarch phosphate (E 1412), phosphated distarch phosphate (E 1413), acetylated distarch phosphate (E 1414), acetylated starch (E 1420), acetylated distarch adipate (E 1422), hydroxypropyl starch (E 1440), hydroxypropyl distarch phosphate (E 1442), starch sodium octenyl succinate (E 1450), acetylated oxidized starch (E 1451), and any combination thereof.

Accordingly, in a preferred aspect, the invention relates to a process for producing crystalline ammonium chloride, wherein the crystallization additive is selected from the group consisting of alginic acid (E 400), sodium alginate (E 401), potassium alginate (E 402), ammonium alginate (E 403), calcium alginate (E 404), propylene glycol alginate (E 405), carrageenan (E 407), gum tragacanth (E 413), gum Arabic (E 414), xanthan gum (E 415), gum karaya (E 416), gellan gum (E 418), gum ghatti (E 419), pectin (E 440i), amidated pectin (E 440ii), cellulose (E 460), methyl cellulose (E 461), ethyl cellulose (E 462), hydroxypropyl cellulose (E 463), hydroxypropyl methyl cellulose (E 464), ethyl methyl cellulose (E 465), carboxymethyl cellulose (E 466), cross-linked sodium carboxymethyl cellulose (E 468), enzymatically hydrolyzed carboxymethyl cellulose (E 469) and any combination thereof.

A preferred heteropolysaccharide is a heteropolysaccharide having a COOH group, a derivative thereof or a heteropolysaccharide having a OSO₃ group.

Accordingly, in a preferred aspect, the invention relates to a process for producing crystalline ammonium chloride, wherein the crystallization additive is a heteropolysaccharide having COOH groups, a derivative thereof, a heteropolysaccharide having OSO₃ groups, preferably a heteropolysaccharide based on galacturonic acid, mannuronic acid, guluronic acid, glucuronic acid or a derivative thereof, cellulose, or a cellulose ether.

Especially, the crystallization additive is a heteropolysaccharide based on galacturonic acid, mannuronic acid, guluronic acid, glucuronic acid or any derivative thereof.

A particular preferred crystallization additive is selected from the group consisting of alginic acid (E 400), sodium alginate (E 401), potassium alginate (E 402), ammonium alginate (E 403), calcium alginate (E 404), propylene glycol alginate (E 405), carrageenan (E 407), gum tragacanth (E 413), gum Arabic (E 414), xanthan gum (E 415), gum karaya (E 416), gellan gum (E 418), gum ghatti (E 419), pectin (E 440i), amidated pectin (E 440ii) and any combination thereof.

More preferred is a crystallization additive selected from the group consisting of alginic acid (E 400), sodium alginate (E 401), ammonium alginate (E 403), gellan gum (E 418), pectin (E 440i), amidated pectin (E 440ii) and any combination thereof.

Most preferred is a crystallization additive selected from the group consisting of pectin (E 440i), amidated pectin (E 440ii), and any combination thereof.

The crystallization additive is usually added in an amount of from 0.1 to 1000 ppm by weight (0.00001 to 0.1 wt %), based on the total amount of NH₄Cl, preferably 1 to 500 ppm by weight, more preferably 5 to 300 ppm by weight. The total amount of NH₄Cl is the calculated value on 100% yield of crystalline NH₄Cl.

Accordingly, in a preferred aspect, the invention relates to a process for producing crystalline ammonium chloride, wherein the amount of the crystallization additive is of from 0.1 to 1000 ppm by weight, based on the total weight of the crystalline ammonium chloride, preferably 1 to 500 ppm by weight.

Increasing the amount of the crystallization additive above the maximum level usually leads to adverse effects on the desired crystal morphology.

Reducing the amount of the crystallization additive below the minimum level also leads to an adverse effect on the desired crystal morphology, for example, by obtaining a more dendritic-like crystal morphology which may result in more intermeshed particles and baking.

The yield of the obtained crystalline ammonium chloride is nearly quantitative, especially ≥98%, in particular, ≥99%.

The median diameter (d₅₀) of the particles of the crystalline ammonium chloride, obtainable by the process, may be in the range of from 50 μm to 1 mm, after the usual removal of fines and oversized lumps. The median diameter may be adjusted within the limits, dependent on the desired median diameter.

Accordingly, in a preferred aspect, the invention relates to a process for producing crystalline ammonium chloride, wherein the crystalline ammonium chloride has a median diameter d₅₀ of from 50 μm to 1 mm.

A suitable method of adjusting the desired particle diameter may be screening, for example, by inline screening within the crystallization loop or by adjusting suitable parameter, like the circulation volume, milling of large crystals, seeding by adding smaller crystals or by targeted reduction of supersaturation.

The crystalline ammonium chloride may be treated with an anticaking agent, stored, filled and packaged. An anticaking agent may be added after the crystalline ammonium chloride is separated and dried, in order to prevent formation of lumps or sizable agglomerates, which may be obtained during storage. Examples of an anticaking agent include calcium phosphate, calcium carbonate, magnesium carbonate, silicon(IV)-oxide or another customary anticaking agent. Preferably, the anticaking agent has an approval as a food additive and an animal feed additive.

The instant process provides a crystalline ammonium chloride, wherein the crystallization additive is attached to the surface of the crystals. Thus, the instant process provides a surface-modified crystalline ammonium chloride.

Accordingly, in a preferred aspect, the invention relates to a process for producing crystalline ammonium chloride, wherein the crystalline ammonium chloride is surface-modified by the crystallization additive.

In a further aspect, the invention relates to a surface-modified, crystalline ammonium chloride, obtainable by a process, as defined in any aspect herein.

The crystallization additive added to the instant process may be present at least partly or completely in the final product, generally in an amount of at least 30 wt %, based on the total weight of the pectin added to the process, preferably of at least 40 wt %, more preferably 40 to 90 wt %. The surface-modified crystalline ammonium chloride may comprise the crystallization additive in an amount of from 0.03 to 1000 ppm by weight, based on the crystalline ammonium chloride, preferably from 1 to 500 ppm by weight, more preferably from 5 to 300 ppm by weight.

The crystalline ammonium chloride, obtainable by the instant process, may suitably be used in applications in the food industry, animal feed industry, for example, as zoo-technical additive, cosmetic industry or pharmaceutical industry.

Accordingly, in a further aspect, the invention relates to the use of a crystalline ammonium chloride, obtainable or obtained by a process, as defined in any aspect herein, as a flavoring agent, as an animal feed additive, as an additive for a cosmetic composition or as an additive for a pharmaceutical composition, preferably as a flavoring agent or as an animal feed additive.

In a further aspect, the invention relates to a process of applying crystalline ammonium chloride, obtainable by a process, as defined in any aspect herein, as a flavoring agent to a food or as an additive to an animal feed, to a cosmetic composition or to a pharmaceutical composition, preferably as a flavoring agent to a food or as an additive to an animal feed.

The process may be carried out with a chemical production unit comprising zones for the reaction stage, the crystallization stage and the separation stage.

Accordingly, in a further aspect, the invention relates to a chemical production unit for carrying out a process, as defined in any aspect herein, the chemical production unit comprising
a reaction zone comprising
   an inlet means (3) for feeding NH₃ (1) into the reaction zone,
   an inlet means for feeding HCl (2) into the reaction zone,
   a mixing device (4),
   a device for phase separation (5) for obtaining an aqueous NH₄Cl solution and a reaction vapor (11),
   a circulating pump (7),
   a heat exchanger (8), preferably provided with vapor (9) during a start-up period,
   a means for passing the aqueous NH₄Cl solution (10) to the crystallization zone,
   a means for passing the reaction vapor (11) to the crystallization zone,
a crystallization zone comprising
   an inlet means for feeding the aqueous NH₄Cl solution into the crystallization zone,
   an evaporator (12) for obtaining an aqueous NH₄Cl-containing suspension,
   a circulating pump (13),
   a heat exchanger (14),
   a means for passing the NH₄Cl-containing suspension (17) to the separation zone,
a separation zone comprising
   one or more separating devices for obtaining crystalline NH₄Cl, and
one or more inlet means (23) for feeding a crystallization additive.

Preferably, the chemical production unit comprises additionally
a droplet separating device (6), preferably a demister,
a pump (16) for withdrawing a part of the aqueous NH₄Cl-containing suspension
a drying means (21),
a container (20) for collecting the mother liquid, and
a means for passing the mother liquid (24) into the reaction zone.

The crystallization zone comprises preferably one or more crystallization loops, more preferably one to three or one to two crystallization loops, more preferably arranged in parallel.

FIG. 1 shows a schematic overview of a preferred embodiment of the process of the invention, comprising a reaction stage, a crystallization stage and a separation stage.

The reaction zone of the reaction stage includes a reaction loop comprising an inlet means (3) for feeding NH₃ (1), a mixing device (4), where HCl (2) is fed, a device for phase separation (5), a droplet separating device (6), a circulating pump (7) and a heat exchanger (8), which is operated with vapor (9) during the start-up phase and any condensate may be collected separately.

The crystallization zone of the crystallization stage includes a crystallization loop comprising an evaporator (10), a circulation pump (13) and a heat exchanger (14). An aqueous NH₄Cl solution (10) is partially removed to be fed into the crystallization loop. The reaction vapor (11) obtained in the device for phase separation (5) is fed into the heat exchanger of the crystallization loop and crystallization zone, resp. Any condensate leaving the heat exchanger may be collected separately.

An aqueous NH₄Cl-containing suspension (17) is partially removed via a pump (16) to be fed into the separation zone. The crystallization vapor (15) obtained in the evaporator (10) is discharged, for example, for further use or collecting as condensate.

The separation zone of the separation stage includes a hydrocyclone (18), a centrifuge (19), wherein both are connected to a container collecting mother liquid (20), and a drying means (21), where crystalline ammonium chloride (22) as final product is obtained. The mother liquid (24) is passed to the reaction loop.

Further, the crystallization additive may be added at various locations, for example, referenced with (23).

In case of two parallel crystallization loops, each of the stream of vapor (9) and the stream of the aqueous NH₄Cl solution (10) is divided into two equal streams.

### LIST OF REFERENCE SYMBOLS

1 NH₃
2 HCl
3 inlet means for feeding NH₃
4 mixing device
5 device for phase separation
6 droplet separating device
7, 13 circulating pump
8, 14 heat exchanger
9 vapor
10 aqueous NH₄Cl solution
11 reaction vapor
12 evaporator
15 crystallization vapor
16 pump
17 aqueous NH₄Cl-containing suspension
18 hydrocyclone
19 centrifuge
20 container for mother liquid
21 drying means
22 crystalline NH₄Cl
23 crystallization additive
24 mother liquid

The instant process provides economical as well as environmental advantages.

The instant process provides crystalline ammonium chloride in approximately quantitative yield, for example, in a very high yield of ≥99% or higher, based on the amount of HCl.

The instant process allows for a heat-integrated process. The crystalline ammonium chloride may be obtained by a two-stage production process, wherein the reaction stage and crystallization stage are separated, but energetically linked. This allows an essentially autothermic process, wherein the energy generated in the reaction stage provides the necessary energy and energy, resp., for evaporation during the crystallization stage. The input of energy generated from the reaction enthalpy of the neutralization reaction in step a) is sufficient to completely evaporate the water introduced with the aqueous HCl. Thus, the reaction enthalpy may be used to form the reaction vapor as well as to form the crystallization vapor, and it may be used for a further process requiring heat, for example, a further evaporation.

As the neutralization reaction and the removal of energy by formation of water vapor within the reaction stage are separated, it is possible to generate reaction vapor, essentially free from NH₄Cl, HCl and NH₃.

A little of entrainment of ammonia, hydrochloric acid and ammonium chloride in the exhaust gas of the reaction and the crystallization stages lead to a minimum loss of starting materials and ammonium chloride via wastewater. Due to the course of the reaction step and the additional purification of reaction and crystallization vapor, condensates of high purity, i.e., generally of low electrical conductivity, are obtained and can be used in other processes. Therefore, any emissions of NH₃, HCl and NH₄Cl in wastewater may be reduced to a minimum.

The application of a slight overpressure during the reaction of NH₃ and HCl may avoid the simultaneous presence of gaseous ammonia and steam which reduces loss of ammonia via the gas phase. Thus, there is also a minimum of loss of ammonia and ammonium chloride via exhaust air.

Further, the instant process enables the preparation of crystalline ammonium chloride in a reliable manner, with a minimum of disruptions caused by possible deposits of salts.

The instant process provides ammonium chloride in crystalline form with a high purity, especially the process is useful for controlling the crystallization of ammonium chloride.

Under the action of the crystallization additive, the ammonium chloride crystals exhibit a favorable morphology, especially a more spherical shape, compared to crystals of ammonium chloride without a crystallization additive, which are characterized by a dendritic crystal structure as well as to crystals of ammonium chloride, described in the prior art.

The crystalline product has a uniform crystal structure having a low specific surface area which is of advantage in that the crystals may be easily, essentially completely and fast dried. Further, the crystalline product shows less agglomeration.

The crystalline product has a narrow crystal size distribution, wherein a narrow crystal size distribution has usually an advantageous effect on product parameters as free-flowing properties, hygroscopicity, bulk density etc.

The crystalline ammonium chloride, as obtained, is usually a flowable powder, also after storage for several months, for example, up to one year or even longer.

All percent, ppm or comparable values refer to the weight with respect to the total weight of the respective basis except where otherwise indicated. The terms “% by weight” and “wt %” are used herein synonymously.

The following examples shall further illustrate the present invention without restricting the scope of this invention.

### EXAMPLE

### 1^{st} Stage: Reaction of NH₃ and HCl

The reaction of ammonia with hydrochloric acid was carried out in a reaction loop containing a phase separator, a circulating pump, a tube bundle heat exchanger, an ejector (jet pump) and a static mixer. An aqueous solution of ammonium chloride (˜40 wt %) was circulated with the pump at a temperature of 95 to 105°. The required amount of gaseous ammonia was dissolved into the reaction loop via the jet pump applying a circulation volume of about ˜500 m³/h.

Subsequently, hydrochloric acid (37 wt %, 0.99 equivalents referred to the amount of ammonia) were added to the reaction mixture using the static mixer maintaining a pressure of 2 bar. The neutralization reaction was already complete with entry into the phase separator. Ammonia and hydrochloric acid were dosed into the reaction loop such that a small excess of ammonia (˜1 mol %) was always added. The temperature of the solution in the static mixer increased from about 98 to 106° C. The reaction solution was dosed into the phase separator via a throttle valve to prevent boiling during the actual reaction. After the valve the reaction solution was boiled by pressure reduction (˜620 mbar), wherein a phase separation took place into aqueous NH₄Cl solution (cooled to ˜100° C.) and vapor.

Water was evaporated from the reaction solution in the phase separator, wherein the temperature of the reaction solution decreased in the phase separator accordingly. The amount of water to be evaporated during the reaction was such the NH₄Cl solution in the reaction loop has a concentration of ˜40 wt % at 100° C. Any solution was removed from the vapor via an aerosol separator and a droplet separator. The vapor was then transferred to a heat exchanger of the subsequent crystallization stage.

The reaction solution passed from the phase separator to the suction side of the circulation pump and was conveyed to the ammonia ejector to close the loop. On the suction side of the circulation pump, ammonium chloride mother liquor from the separation stage was added.

The heat exchanger, installed between the circulating pump and the ammonia ejector, was used to heat the reaction mixture to the operating temperature of approx. 100° C. with steam of 2 bar when starting up the chemical production unit.

On the pressure side of the pump, part of the reaction solution was constantly removed from the reaction loop and fed to two crystallization stages for further evaporation.

### 2^{nd} Process Stage: Crystallization

The crystallization and evaporation of the ammonium chloride solution were carried out in two parallel circulating evaporators. Only one is described in the following.

The crystallization loop contains a circulating pump, a tube bundle heat exchanger and an evaporator. Crystallization and evaporation were carried out with a high circulation volume at a temperature of ˜55° C. and a pressure of 110 mbar. First, the circulation volume was conveyed from the pump to the heat exchanger, which was operated with a part of the vapor of the reaction stage. The solution was heated in the heat exchanger by ˜1 to 2° C. In case of forming condensate, this may be discharged in a separate container.

Prior to the heated solution was fed into the evaporator, the reaction solution of the 1^{st} stage was dosed into the crystallization loop. Water was evaporated from the solution in the evaporator leading to crystallization of ammonium chloride, wherein a suspension of ammonium chloride (˜20 wt %) was moved. The vapor resulting from evaporation was condensed after separating solution therefrom by a droplet separator. The vacuum required for evaporation was generated with a liquid ring compressor.

Between the evaporator and the circulating pump, part of the ammonium chloride suspension was constantly removed and fed into the separation stage. The pre-dissolved crystallization aid may be dosed into the crystallization loop.

### 3^{rd} Process Stage: Separation

The discharge of the two evaporators was first passed into several hydrocyclones to be separated into a thickened lower flow and an upper flow having a lower solids content. The combined upper flows were fed into a mother liquor container. The combined thickened lower flows were directed to a pusher centrifuge. The crystallization additive may also be dosed prior to the pusher centrifuge.

The moist ammonium chloride separated as centrifugate was dried. The saturated ammonium chloride solution separated as centrate was discharged into the ammonium chloride mother liquor container. The mother liquor was pumped into the reaction loop.

The dried ammonium chloride was sieved to remove lumps and stored in bags. The product is obtained as a flowable powder in a yield of >99%.

FIG. 2 shows a microscope picture of the product obtained by the Example, using 50 ppm by weight of pectin, added to the aqueous solution of ammonium chloride in the crystallization loop.

Based on the analysis of the total organic carbon (TOC) of the mother liquid, it was determined that 75% of the added pectin are attached to the final product. TOC was determined according to DIN EN 1484:2019-04.

1.-27. (canceled)

28. A process for producing crystalline ammonium chloride in the presence of a crystallization additive, the process comprising the steps
a) reacting NH₃ and HCl by feeding NH₃ and HCl to an aqueous NH₄Cl solution;
b) crystallizing ammonium chloride from an aqueous NH₄Cl solution obtained in step a), and
c) separating the crystalline ammonium chloride, wherein the energy required in step b) is generated in step a).

29. The process according to claim 28, wherein the aqueous NH₄Cl solution of step a) is run through a reaction loop.

30. The process according to claim 28, wherein NH₃ is fed as a gas and HCl is fed as an aqueous solution having a concentration of from 20 to 38 wt %.

31. The process according to claim 28, wherein the aqueous NH₄Cl solution has a concentration of 30 to 45 wt % in step a).

32. The process according to claim 28, wherein the mole ratio of HCl:NH₃ is of from 0.95:1.05 to 1.05:0.95.

33. The process according to claim 28, wherein step a) is conducted under a pressure of 0.1 to 3 bar and a temperature of from 50 to 130° C.

34. The process according to claim 28, wherein the energy required in step b) is generated in step a) by the heat of reaction of reacting NH₃ and HCl, which is released in form of reaction vapor.

35. The process according to claim 28, wherein step a) contains
a-1) a step of reacting NH₃ and HCl and
a-2) a step of generating reaction vapor,
wherein said steps are carried out separately.

36. The process according to claim 28, wherein the reaction vapor is generated by a pressure reduction.

37. The process according to claim 28, wherein the reaction vapor is purified.

38. The process according to claim 28, wherein a part of the aqueous NH₄Cl solution obtained in step a) is fed to step b).

39. The process according to claim 28, wherein the crystallization in step b) is conducted by one or more evaporative crystallization loops.

40. The process according to claim 28, wherein the crystalline ammonium chloride is separated in one or more separation stages.

41. The process according to claim 28, wherein a mother liquid obtained in step c) is recycled to step a).

42. The process according to claim 28, wherein the crystallization additive is added in step b) and/or step c).

43. The process according to claim 28, wherein the amount of the crystallization additive is of from 0.1 to 1000 ppm by weight, based on the total weight of the crystalline ammonium chloride.

44. The process according to claim 28, wherein the crystallization additive is a polysaccharide, a derivative thereof or any combination thereof.

45. The process according to claim 28, wherein the crystallization additive is selected from a heteropolysaccharide, a derivative thereof, cellulose, a derivative thereof and any combination thereof.

46. The process according to claim 28, wherein the crystallization additive is a heteropolysaccharide having COOH groups, a derivative thereof, a heteropolysaccharide having OSO₃ groups, cellulose, or a cellulose ether.

47. The process according to claim 28, wherein the crystallization additive is selected from the group consisting of alginic acid (E 400), sodium alginate (E 401), potassium alginate (E 402), ammonium alginate (E 403), calcium alginate (E 404), propylene glycol alginate (E 405), carrageenan (E 407), gum tragacanth (E 413), gum Arabic (E 414), xanthan gum (E 415), gum karaya (E 416), gellan gum (E 418), gum ghatti (E 419), pectin (E 440i), amidated pectin (E 440ii), cellulose (E 460), methyl cellulose (E 461), ethyl cellulose (E 462), hydroxypropyl cellulose (E 463), hydroxypropyl methyl cellulose (E 464), ethyl methyl cellulose (E 465), carboxymethyl cellulose (E 466), cross-linked sodium carboxymethyl cellulose (E 468), enzymatically hydrolyzed carboxymethyl cellulose (E 469) and any combination thereof.

48. The process according to claim 28, wherein the crystalline ammonium chloride is surface-modified by the crystallization additive.

49. The process according to claim 28, wherein the crystalline ammonium chloride has a median diameter d₅₀ of from 50 μm to 1 mm.

50. A surface-modified, crystalline ammonium chloride, obtained by the process according to claim 28.

51. A flavoring agent, animal feed additive, additive for a cosmetic composition or additive for a pharmaceutical composition comprising a crystalline ammonium chloride, obtained by the process according to claim 28.

52. A process of applying crystalline ammonium chloride, obtained the process according to claim 28, as a flavoring agent to a food or as an additive to an animal feed, to a cosmetic composition or to a pharmaceutical composition.

53. A chemical production unit for carrying out the process according to claim 28, the chemical production unit comprising
a reaction zone comprising
an inlet means for feeding NH₃ into the reaction zone,
an inlet means for feeding HCl into the reaction zone,
a mixing device,
a device for phase separation for obtaining an aqueous NH₄Cl solution and a reaction vapor,
a circulating pump,
a heat exchanger,
a means for passing the aqueous NH₄Cl solution to the crystallization zone,
a means for passing the reaction vapor to the crystallization zone,
a crystallization zone comprising
an inlet means for feeding the aqueous NH₄Cl solution into the crystallization zone,
an evaporator for obtaining an aqueous NH₄Cl-containing suspension,
a circulating pump,
a heat exchanger,
a means for passing the NH₄Cl-containing suspension to the separation zone,
a separation zone comprising
one or more separating devices for obtaining crystalline NH₄Cl, and
an inlet means for feeding a crystallization additive.

54. The chemical production unit according to claim 53, additionally comprising
a droplet separating device,
a pump for withdrawing a part of the aqueous NH₄Cl-containing suspension,
a drying means,
a container for collecting the mother liquid, and
a means for passing the mother liquid into the reaction zone.
